(19) United States
(12) Patent Application Publication
(10) Pub. No.: US 2024/0325156 A1
(43) Pub. Date: Oct. 3, 2024

(54) APPARATUS AND METHOD FOR UNIFORM COATING OF IMPLANTS WITH ANTIBIOTIC CEMENT

(71) Applicant: Genesis Medical Devices LLC, Indialantic, FL (US)

(72) Inventors: Daniel Nick Segina, Satellite Beach, FL (US); James A. Proctor, JR., Indialantic, FL (US)

(21) Appl. No.: 18/599,400

(22) Filed: Mar. 8, 2024

Related U.S. Application Data
(60) Provisional application No. 63/450,918, filed on Mar. 8, 2023.

(51) Int. Cl. A61F 2/30; A61B 17/72

(52) U.S. Cl. CPC A61F 2/30942; A61B 17/72; A61F 2002/30784; A61F 2310/00005

(57) ABSTRACT

A preconfigured, customizable intraoperative mold for the assembly of implants having uniform coating(s). One or more plugs are adapted to prevent incursion of coating material into engineered or other openings in the implant. The dimensions of these plugs, or other standoffs, is chosen to control spacing of the implant away from the interior surfaces of the mold. The plugs and/or standoffs thus further control a thickness, profile, distribution, types, or other configuration of the coating materials.

### CROSS REFERENCE TO RELATED APPLICATION(S)

This patent application claims priority to a co-pending U.S. Provisional Patent Application Ser. No. 63/450,918 entitled “APPARATUS AND METHOD FOR UNIFORM COATING OF IMPLANTS WITH ANTIBIOTIC CEMENT” filed on Mar. 8, 2023, the entire contents of which are hereby incorporated by reference.

### BACKGROUND

An intramedullary rod, also known as an intramedullary nail (IM nail) or inter-locking nail or Küntscher nail, is a cylindrical metal rod forced into the medullary cavity of a bone. Intramedullary nails have been used for many years to treat fractures of long bones of the body. A significant problem with early designs of these implants was their failure to prevent bone collapse or rotation in inherently unstable fractures. This has been addressed by ‘locking’ the nail in place, where bolts on each end of the nail fix it to the bony cortex, preventing rotation among bone fragments. This has led to the emergence of locked IM nails as a product, which is the standard today. Locking features are provided by engineered openings in the nail, such as threaded holes, where stabilization screws are used to attach the nail to the bone. Threaded features in the nail can also be used to attach the nail to external fixator structures such as outriggers to treat certain types of complex fractures or for bone lengthening or straightening.

Antibiotic cement-coated nails or rods are used for internal fixation or repair of infected bones, also called osteomyelitis. A mixture of orthopedic cement, such as polymethyl methacrylate (PMMA), similar to what is used to hold a knee replacement in place, is prepared. This viscous, play-dough like substance is placed onto the surface of the nail, and allowed to cure. Once hardened, the coated rod is then inserted into the bone in the same way a non-coated rod is placed, with the now added benefit of carrying local antibiotic to treat infection.

See as one example: Liu, Jane Z. et al. in “Coated nails: is their use supported by the literature?”, OTA International 4 (3S): p. 110, June 2021. Antibiotic-coated intramedullary nails are especially useful in treating diaphyseal infections requiring stability, such as those involving fractures and nonunions. The nails are made by injecting antibiotic-impregnated polymethylmethacrylate or “cement” around a metal core using a silicone tube as a mold. There are a variety of techniques that can be used to customize the nail to the affected site.

DePuy Synthes sells a product they call PROtect™ which consists of a pre-manufactured nail coated with an antibiotic agent. This product is limited to just the vendor's own available nail designs. It does not allow for customization or accommodation of implantable devices manufactured by other vendors.

Other products, called “spacers”, are a temporary prothesis which is itself made of bone cement, and impregnated with antibiotics. These are fully pre-manufactured. They can serve as a temporary replacement prosthesis, such as an artificial joint for total knee arthroplasty used to resolve, infection, a primary arthroplasty. A benefit is that a spacer can be fabricated to replace any manufacturer's implant. When the implant has become infected, the spacer can be temporarily used during resolution of the infection, and then removed when a new permanent prothesis is available.

### SUMMARY

There are several problems with existing processes and products.

“One off” applications such as spacers are inconsistent and non-controllable. They involve a “fiddle factor” associated with cumbersome and disorganized assembly processes. The resulting success is on an ad-hoc basis, and largely depends upon the skill of and the individualized techniques employed by the particular surgeon

These existing approaches also result in variation in the distribution of antibiotic coatings. Ideally, a minimum consistent thickness of the coating is achieved in all areas of the nail to prevent flaking and/or debris generation, coating fragmentation and/or separation of the coating. During later removal of the implant, this debris is difficult to remove, and may be problematic. Another result is inconsistency or unknown/uncontrolled distribution of an antibiotic within the cement compound surrounding the nail or other implant.

The nail itself can be adversely affected. Pre-engineered features such as threaded openings are often provided in intramedullary nails to accommodate locking screws, fixator structures, outriggers, and other apparatus. Prior art coating processes are prone to leaving these openings partially or wholly filled with compound or other agents.

Pre-configuration of implants prior to the inter operative situation is generally not feasible due to stability issues, further preventing temporary storage of apparatus prior to the presence of the operative patient.

Nail coating processes also drive the need for additional operating room time. People other than the surgeon are also needed, such as anesthesiologists and other operative personnel. These can significantly add to operating room costs (estimated to range from $19 to $131 or more per minute.) See Childers, Christopher P., “Understanding costs in the operating room”, JAMA Surg. 2018; 153 (4) Apr. 18, 2018.

Excluding the cost of anesthesia, the anesthesiologist, professional surgeon and other support personnel, the typical process for coating a single implant (such as a nail) can take from 15 to 20 minutes depending on implant type and anatomical location. This cost of the configuration procedure (not including materials) is at least $750 plus the cost of the surgeon's own time. Other expected and unexpected time includes additional time needed for cement curing or exothermic waste heat issues (such as when an antibiotic gets too hot and de-natures) further impacting total costs.

Some solutions exist to reduce heating due to setting processes, based upon slowing the curing process. However this, too, has a negative effect on the time needed to perform configuration of the implant and compound.

The result is additional operating room personnel cost and patient anesthesia time, not to mention materials waste due to complex, multi-step configuration process.

What is needed are devices, apparatus and methods that can provide improved consistency and control over the fabrication of coated intramedullary nails and other implantable devices, such as plates or other structures. Coating processes should apply antibiotics and other agents uniformly and without interfering with features of the implant.

These approaches should also result in reducing the time needed for surgical staff, operating room occupancy, and reduced patient anesthesia time.

Furthermore, they should provide the ability to use alternative agents including thermally unstable materials or drugs/compounds. The need to adhere to particular temperature thresholds or durations should also be avoided.

The devices, methods and apparatus should also be adaptable to different implant designs and thus are ideally “manufacturer agnostic” while being straightforward to implement, easily repeatable, less prone to error, and requiring only moderate surgical skill.

The resulting techniques should also be more environmentally sustainable, and reduce waste by being more efficient and environmentally friendly.

### BRIEF DESCRIPTION OF THE DRAWINGS

FIGS. 1A-1B illustrate an example of a standoff tab or plug that is used to prevent penetration of a coating material within the openings of an implantable device.

FIGS. 1C-1F illustrate threaded portions of the plug in more detail.

FIGS. 2A-2B show assembly of the plugs into an example implantable device such as an intermedullary nail.

FIGS. 3A-3C show one embodiment of a casting sleeve or mold tube in which the implantable device is inserted for coating.

FIGS. 4A-4D are additional illustrations of an example mold tube and implant device.

FIGS. 5A-5D illustrate using a different mold that is formed as an upper and lower half.

FIGS. 6A-6E show a plate-type mold.

### DETAILED DESCRIPTION OF ONE OR MORE EMBODIMENTS

Described herein are various embodiments of a preconfigured, customizable intraoperative mold that enables uniform coating of implant devices as well as processes for assembly and use of the same.

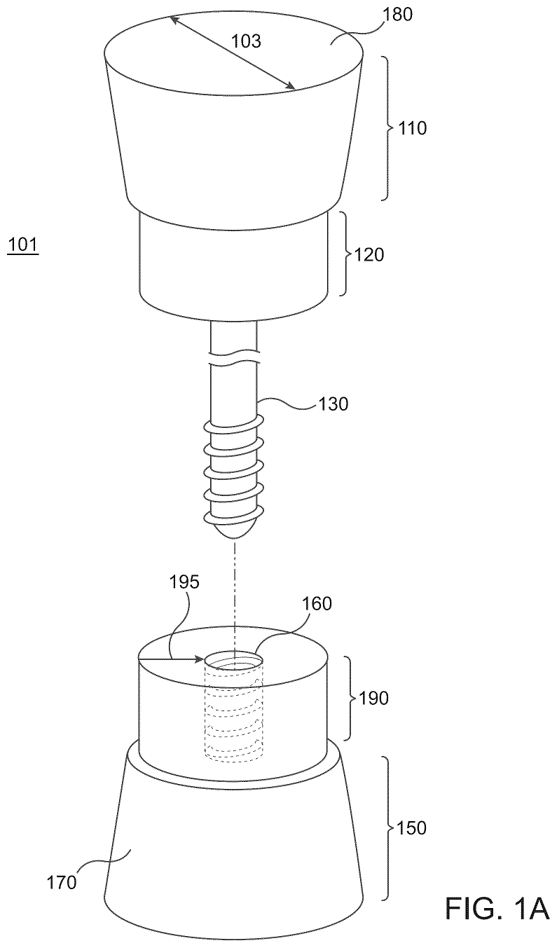

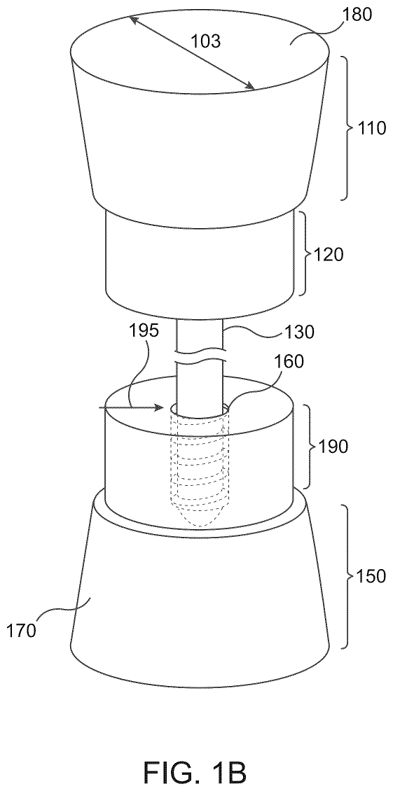

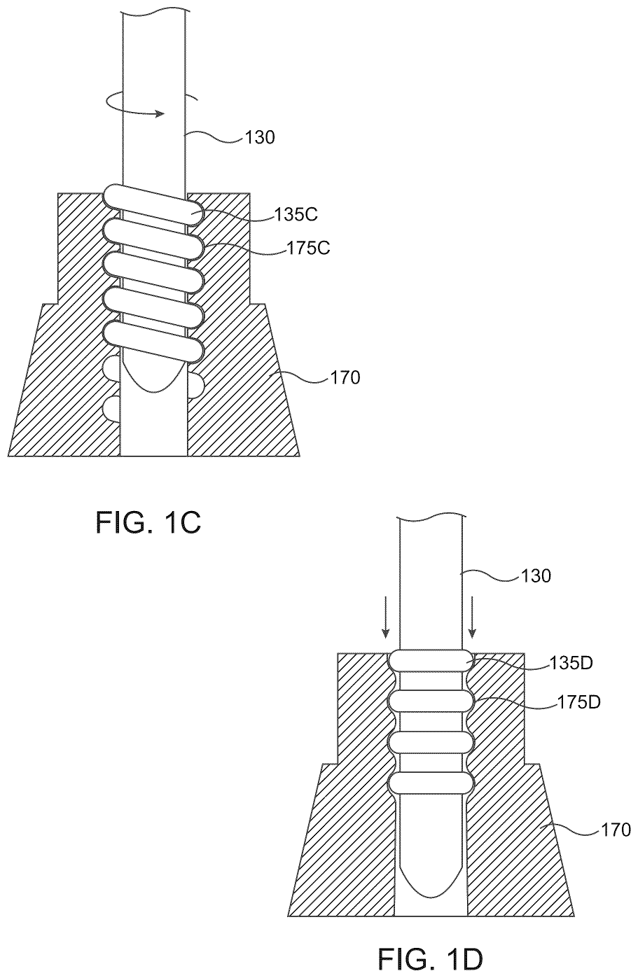

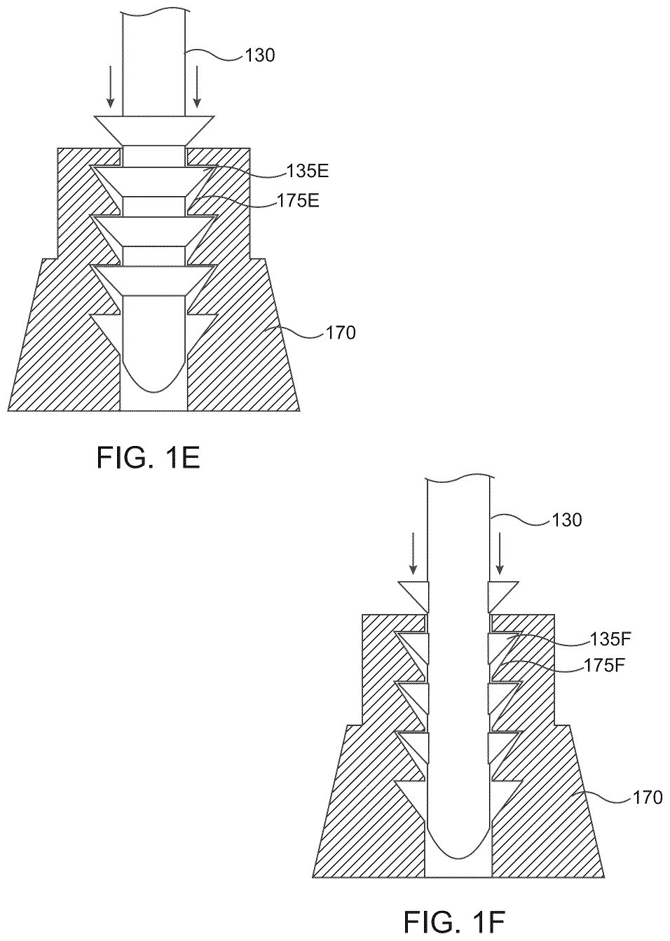

FIGS. 1A and 1B show an example of a standoff tab 101 (also referred to as plug 101 herein) that is designed to prevent penetration of coating material(s) within the openings/apertures of an implantable device. As will be understood in connection with other figures, the height 110 of the plug 101 may be selected to correspond to an appropriately chosen thickness of the coating or mantle to be applied to the implantable device.

In some embodiments, the diameter 103 of the plug 101 tapers from a wide diameter at the outboard end to a narrower diameter at an inboard end, and may include one or more recesses 120. The diameter 103, taper and recesses are chosen to assist with preventing extravasation or leakage of coating material from or within the implantable device.

In some embodiments, the plug 101 may actually consist of two pieces, a first or upper plug portion 180 and second lower or mating plug portion 170. Upper plug portion 180 has a component 130 designed to allows for coupling to an opposed opening 160 in mating plug 170. Mating plug 170 may also have a recessed area 190 and a tapered end portion 150 corresponding to the height 110 of tapered portion and recess 120 of plug portion 180. Inner diameter 195 is chosen to correspond to the thickness or diameter of an opening within the implant into which the plug portion 180 and/or mating plug 170 are inserted.

FIGS. 1C through IF show more details of example of plug 180 and mating plug 170 and how component 130 and opening 160 may be ratcheted or threaded. These various arrangements prevent decoupling of plug 180 and mating plug 170 after assembly. These enlarged drawings of standoff tab/plug 101 show additional detail for an embodiment of the recess 120. These drawings also show an example female opening 160 to accept male component 130 that may be either threaded 135C, 175C (FIG. 1C) or ratcheted using differently shaped ridges such as 135D, 175D (FIG. 1D) or 135E, 175E (FIG. 1E), or 135F, 175F (FIG. 1F). Yet other fastening approaches may be used in other embodiments.

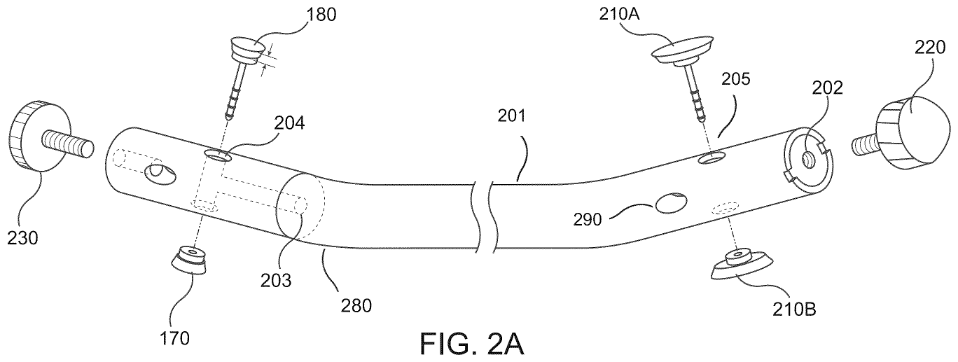

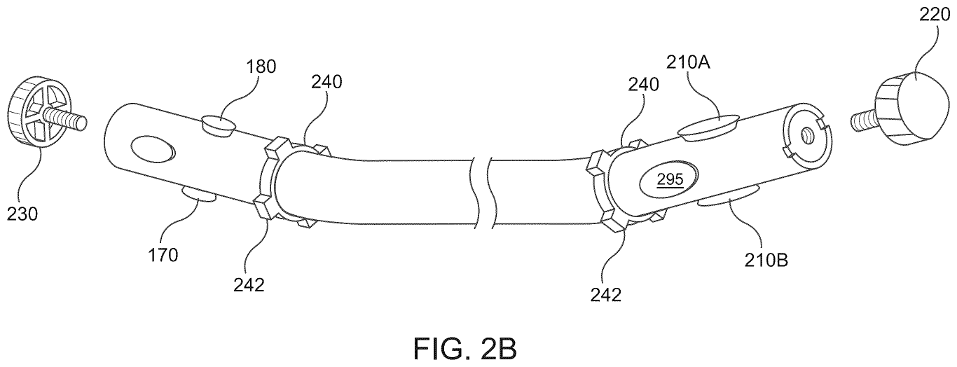

FIGS. 2A and 2B show the plug portion 180 and mating plug 170 being used with an example implantable device 201. The example implantable device 201 may be an intramedullary nail or other medullary fixation device. Yet other types of implantable devices 201 may benefit from the teachings herein. In selected embodiments, an interlocking through-hole 204 is also formed in implantable device 201 into which plug portions 180 and 170 are inserted and assembled together such as by threading, ratcheting or the like. The insertion of plug portions into hole 204 prevents access by the coating material to internal areas 203 of implantable device 201 that would otherwise be possible during the coating process.

Oval-shaped interlocking plugs 210A and 210B are an alternative embodiment of the round plugs 180 and 170. These differently shaped plugs may be used as alternative geometry to match the oval or other noncircular openings 205 in implantable device 201.

The device 201 may also have one or more pre-engineered openings or ports 202 that serve various purposes, such as to enable attachment of screws, fixators, or other instrumentation to the implantable device 201. One or more proximal protection plug 220 are thus provided to protect these openings and/or prevent of inflow of coating material into insertion instrumentation coupling port 202. Protection plug 230 provides protection for a cannulated portion of the implantable device at the distal end.

Other plugs 191 may further protect the distal interlocking ports 202 of the nail 201, and further prevent the inflow of coating material into interlocking ports 202 through hole 204.

The implantable device may have a bend 280, such as the so-called Herzog bend, along its proximal third. Such a bend 280 is designed to enable the nail to enter a medullary canal of the tibia without penetrating the knee joint. While this Herzog bend is only applicable to a tibial nail, other medullary nails may have “bends” or “curves” that are designed to accommodate anatomical constraints of the affected bone. Other embodiments contemplate plates or nails and may include bends or shapes accommodating anatomical constraints as well.

Annular standoffs or o-rings 240 having optional tabs 242 may also fit over the outer diameter of implantable device 201. These rings 240 and tabs 242 assist with correct placement of the implant within the antibiotic coating apparatus, as described below.

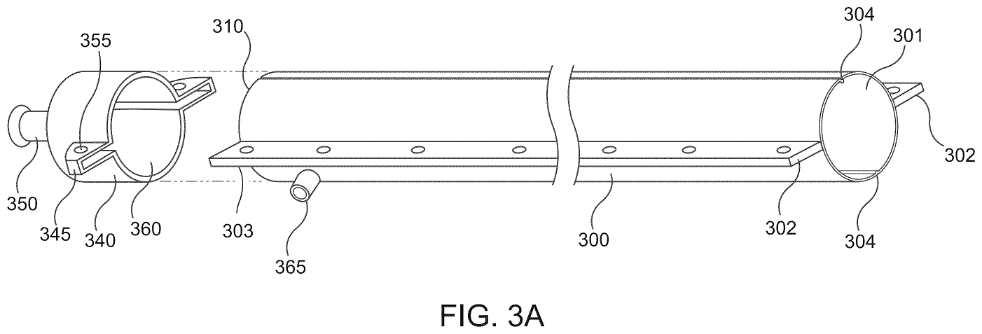

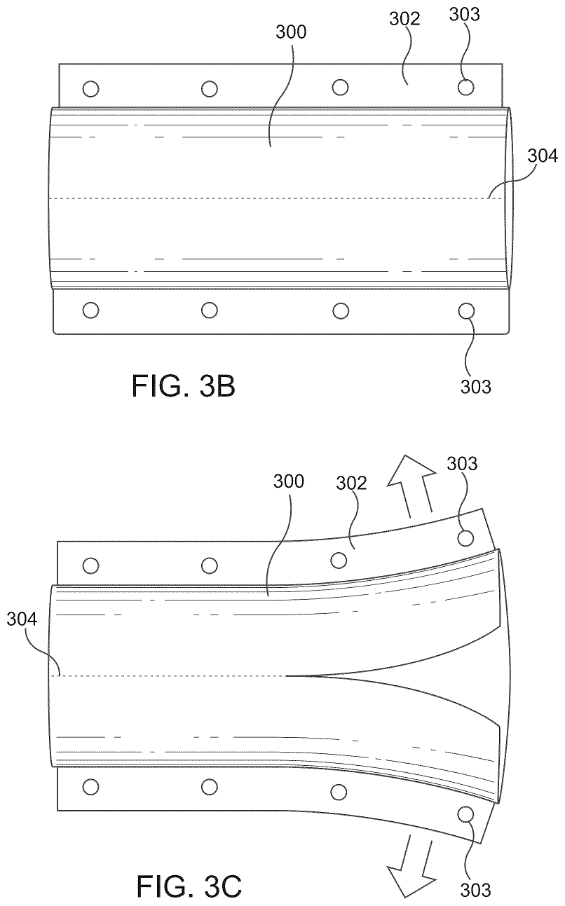

FIGS. 3A-3C show an example casting sleeve or mold, here formed as a tube 300. As discussed in more detail below, tube 300 is typically formed of a flexible or semi-rigid material and provides an opening or cavity into which the implant 201 is inserted for the coating process. The tube 300 is shown in three dimensional profile. An opening port 301 provides for acceptance of the implant 201 for a coating procedure. The coating procedure is preferably carried out in a sterile environment such as an operating room. Recessed/notched zones 304 at the 12:00 o'clock and 6:00 o'clock position are designed to assist in the disassembly of the mold 300 and removal of the mold tube 300 after setting or curing of the implant coating. Mold tube 300 can be trimmable to provide for customization of a desired length during the coating procedure.

Side flanges or tabs 302 noted at the 9:00 o'clock and 3:00 o'clock position are shown with a series of anchoring holes 303 for appropriate attachment of retraction devices for assisting in removal of the implant device 201 after curing/setting of the implant coating. An end cap 340 provides a seal to the casting sleeve/mold tube 300, restricting coating flow past the end portion of the implant 201.

Recessed areas 345 are designed to interface with side tabs 302 and attach through 303 anchoring holes 303 to form an intricate seal. Evacuation port 350 noted at the distal end of end cap 340 allows for the egress of air and/or coating material and promotes more uniform coating. The evacuation port 350 also provides for possible mating with a negative pressure source to assist in the flow of the coating material. One or more holes 355 in end cap 340 may align with holes 303 at end of tube 300 and used with fasteners (not shown) to secure end cap 340 to tube 300. Port(s) 365 may also be located along the length of tube 300 to provide for insertion/injection of coating material.

After the coating process is complete, longitudinal scoring lines or perforation(s) 304 may assist with disassembly or breaking apart of tube 300 for extraction of the implant 201.

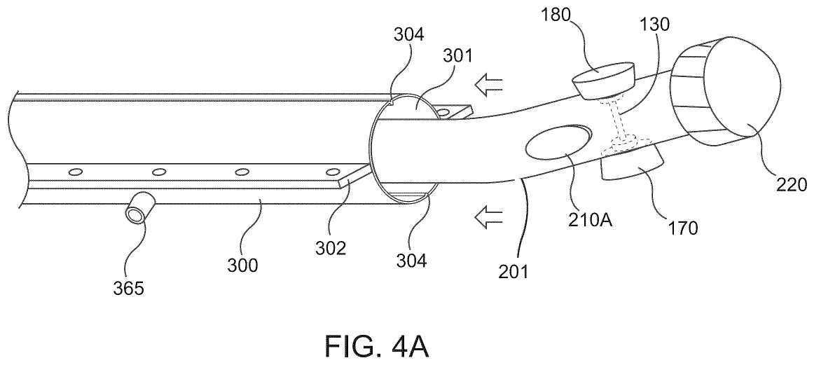

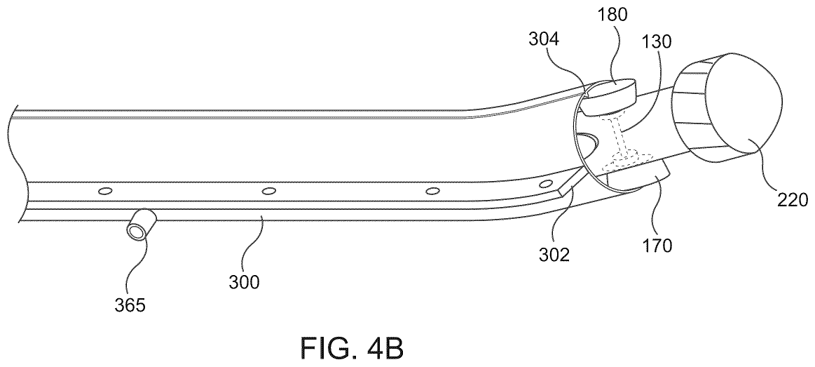

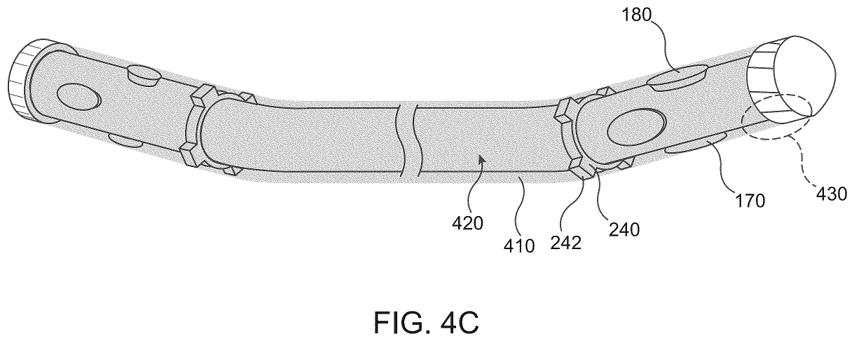

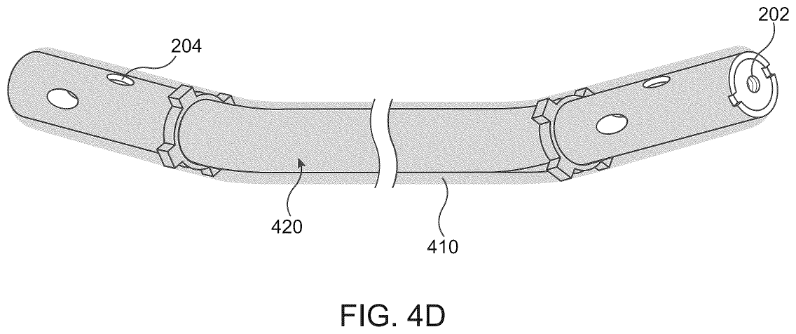

FIGS. 4A-4B depict an example medullary implant 201 being inserted into the casting sleeve/mold tube 300 in the direction of the arrows. Plugs 180 and 170 are shown coupled through the central portion of the implant 201. Plugs 180 and 170 also act as spacers to allow for centering within the casting sleeve/mold tube 300, to thereby produce a uniform circumferential coating.

FIG. 4C shows the implant 201 now fully inserted into the assembled tube 300, with end caps attached. Coating material 410 has now been applied to the outer surface of the implant 201, uniformly from a center portion 420 to an end portion 430.

FIG. 4D is the coated implant 201 now extracted from the tube 300, after the coating process is complete. With end caps 220, 230 removed, implant 201 is now ready for installation, such as into a medullary bone canal.

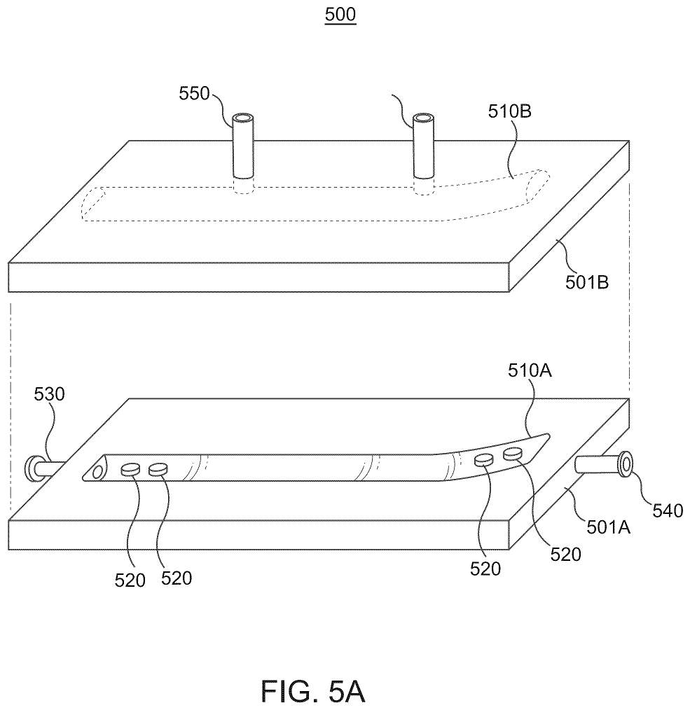

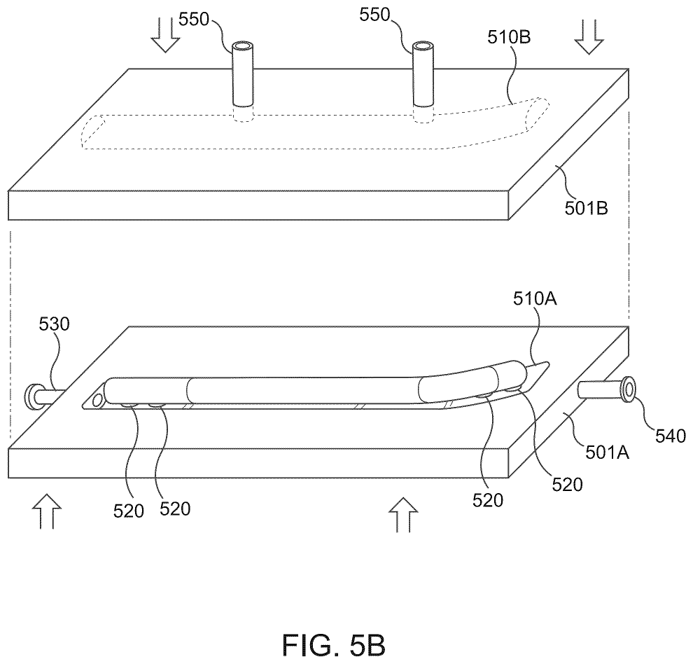

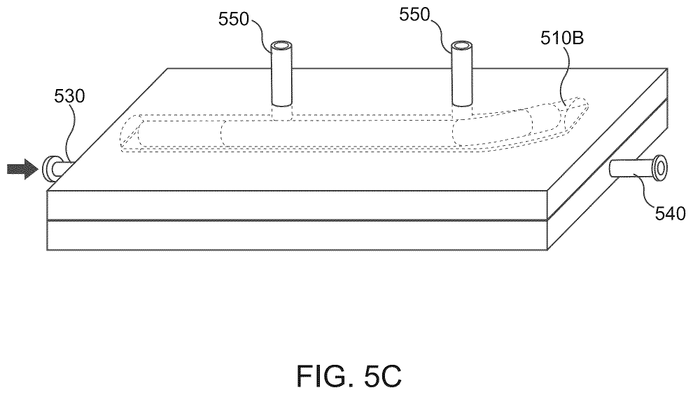

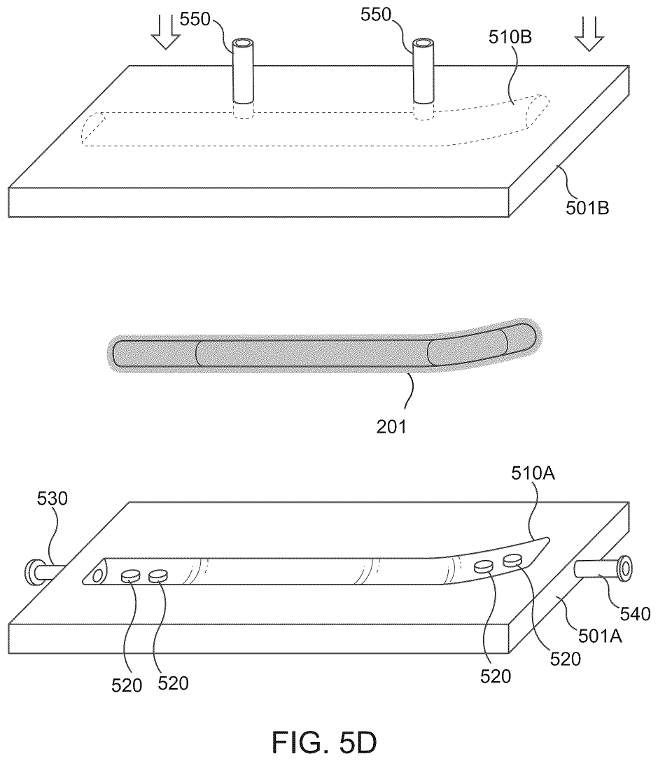

FIGS. 5A-5D are an alternative embodiment of a mold 500, here having a fixed geometry. This type of mold consists of a rigid or semi-rigid lower portion 501A and a mating upper portion 501B. Both mold portions 501A and 501B are designed to provide an internal cavity that surrounds the desired implant 201 chosen for coating. An inflow port 540 provides for the injection of the coating and an outflow port 530 provides for evacuation of coating material and/or air. A negative pressure source can be coupled if desired through the outflow port 530. In other embodiments, such ports may be utilized for the introduction of agents other than, or in additional to, antibiotic cement, as will be discussed in more detail below.

Continuing with the current embodiment, centering standoffs or tabs 520 may be placed within lower portion 501A for raising the implant device 201 above mold surface 510A and/or spacing the implant device 201 away from surface 510B of upper portion 501B, further promoting uniform coating around the implant device 201. Evacuation ports 550, if provided, also assist in a uniform coating application. This type of mold can accommodate a plate in some embodiments. In other embodiments, other types of protheses may be accommodated.

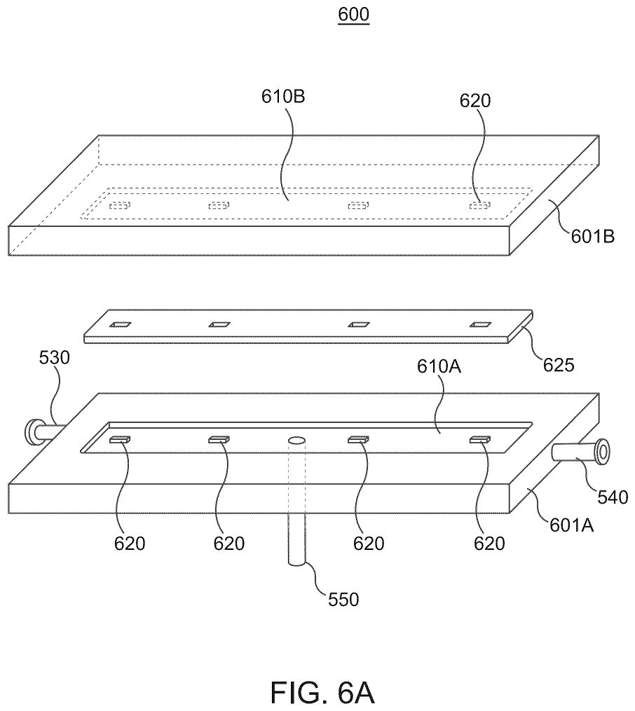

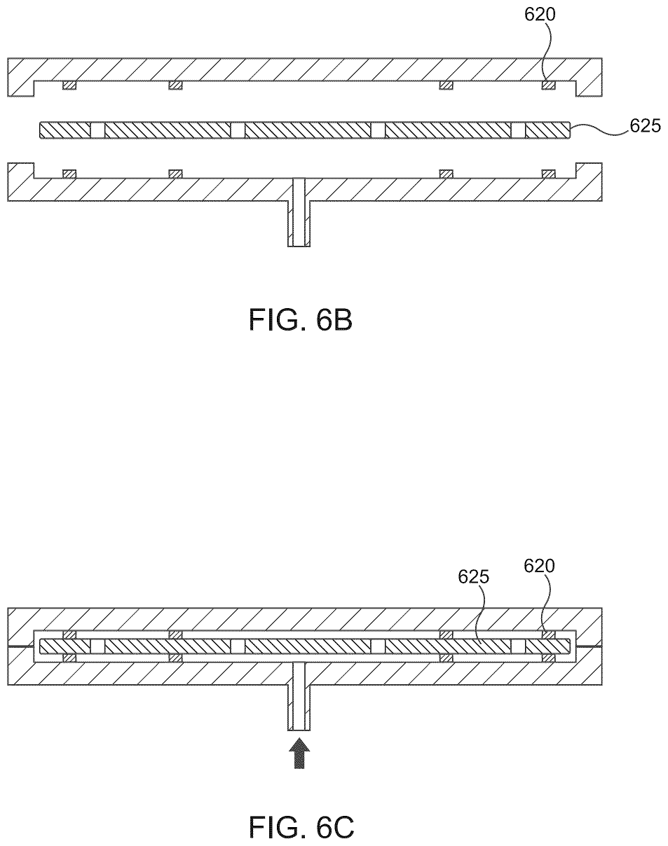

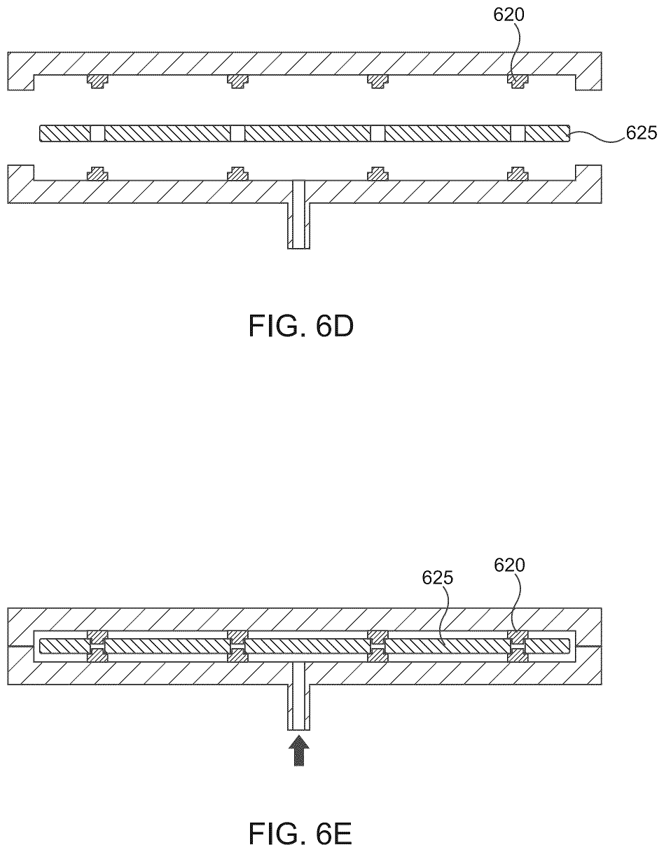

FIGS. 6A-6E are a still further embodiment of a mold 600 designed to accommodate a plate type implant 625. A lower mold portion 601A is sandwiched with upper mold portion 601B to provide a cavity for the plate implant 625. Standoff tab/plug 620, as shown, obstructs screw holes 630 within the plate implant 625 and protects them from inflow of coating material as well as promotes uniform coating. Recessed area 610A with its counterpart matching area 610B conforms to the geometry of the plate implant 625 and produces a uniform coating. Inflow port 540 and outflow port 530, as shown, are provided for the injection of coating material and evacuation of coating material.

### Implementation Options

It will be appreciated by those of skill in the art that various options are possible for implementing the components and processes described herein.

The molds 300, 500 or 600 may be pre-configured for uniform coating of an agent or other compound in an operating room. They may be formed from biocompatible compounds such as polymers, inert or non-absorbable polymers, polymethyl methacrylate (PMMA) or other synthetic resin, bio-absorbable polymers such as polylactic acid (PLA), non-polymers such as calcium salt pyrophosphate or other insoluble or soluble salt materials.

In some embodiments, plugs 101, 170, 180, 210A, 210B, and the like are formed of the same, similar or compatible materials as the molds 300, 500, 600.

A variety of agents can be coated onto or into the implant 201. These may include agents that are thermally stable, or thermally non-stable relative to exothermic polymerization. Agents may include antibiotics such as aminoglycosides or vancomycin. Chemotherapy agents may also be applied. The agents may be biological such as growth factor or stem cell agents. The agents may be ferrous, copper, silver in combination with other agents.

Pain medications, such as locally released lidocaine or other medications can be targeted to selected parts of the implant, allowing for lower overall level medication dosage. For example, medication or chemotherapy or other agents can be used to treat oncological or metabolic conditions of the bone. The agents can be coated in layers with different or alternative time release profiles. In particular, non-homogeneous distribution of agents is possible with differing components of substrate compound material. For example, different antibiotics may be applied with differing time release profiles using PLA or other profile of polymers with different absorption or dissolution rates. Different agents may also be interspersed between different type of PLA layers to result in a coating profile.

The agents may also have different densities targeted to a spatial distribution across a profile of the implant. For example, agent may be concentrated in one location, or agent distribution may be radially or axially or anatomically targeted. An agent may also be packaged within a “bead” that is dropped into or at certain locations as the implantable device is inserted into the tube. As another example densities or concentrations of agents may be injected into the mold so as to correspond to allocation of an anatomical feature concentrating therapeutics in such a locality. One example would be to release a medication at the site of a tumor, concentrating chemotherapy or other agent local to the area, but reducing the concentration of the agent in an area in which therapy is not required or desired.

The implant 201 can include many types of surgical devices including an intramedullary nail, rod, plate, wires for use with structurally sound or intact bones, arthroplasty components, screws, spacers, cages, pins, artificial joints or components thereof, or other components designed to repair, augment, or replace a body part.

The molds may adapt to differently configured devices that are manufactured by a variety of vendors. For example, a mold may be constructed as a negative imprint of a specific device. Or the mold may be modular and assembled from different components to match the shape of a particular implantable device. These approaches thus permit customizing the mold to the geometry of a specific vendor's nail or other implant where the length may be varied depending on the anatomy of the patient.

The size of the mold can be chosen according to a desired coating profile. In one embodiment, a goal may be to coat at least 85% of the nail; however in another application the goal may be to coat 100% of the surface of the nail. An example overall mold diameter may be 12 mm for coating a 10 mm diameter nail.

Customization for different implants may include plugs or inserts of selected sizes and locations. It may be necessary in some instances to accommodate differences in the proximal geometry of the implant such as at the Herzog bend in a tibial nail.

In some instances, a distal bend may not need further accommodation. For example, the thickness of the walls of a flexible mold may be sufficient to avoid contraction (in other words, the material used for the mold may be sufficiently pliable to provide enough “give” around a Herzog bend). However in other instances an annular spacer or insert (such as o-ring 240 as shown in FIG. 4C) may assist with holding the inner surfaces of the mold away from the implant near the location of a bend, and further ensure uniform thickness of the finished coating, while still accommodating the variability in geometry of different manufacturer's implants.

Another approach is to not coat the proximal portion around the Herzog or other bend, including the holes.

Alternatively, the mold may be formed of slightly compressible material such as silicon or a foam. In this instance, and in some other embodiments as well, hard points (such as items 520 in FIG. 5A) are placed within the mold to act as stand-offs and centralize the nail within the mold diameter. These standoffs or tabs can be connected to an embedded support base within such a flexible mold. Alternatively, standoffs can be attached or connected to the nail itself, in addition to or instead of, to the mold. In some instances, three or four points of contact should be provided at each prospective bend in the mold or at other variation points (or more often if the mold material requires more support). This can be advantageous in the presence of forces that may exist from the use of cement in the nail material.

The locations chosen for placement of plugs 170 and 180 and the associated through holes 140 (and/or the diameter of the shaft and grooves used for the plugs) can also assist with accommodating variation in different implant devices.

When the mold is fabricated as a tube of flexible material, it may be fabricated and then shipped as a rolled up tube or condom-like device which is then unrolled over the proximal end of the implant. In this instance, the plugs may be inserted prior to rolling out the tube.

If cement or other material is to be applied to the nail, such materials may be put into the mold first, before the nail is inserted, and the mold then closed.

Alternatively, the nail may be placed the into mold, and then the mold is closed, and the material is injected into mold. In this case, the aforementioned injection and ejection ports should be included and accessible to the closed mold.

As mentioned briefly above, hard points may be included with the mold to set the inner nail geometry. Also, the plugs 170, 180 and/or the rings 240 can serve as “centering tabs” to set the location of the nail within the mold. These centering tabs can be replaceable to accommodate differing nail diameters, or may be trimmable to accommodate differing nail diameters. They can also include marks, or break points that represent pre-measured locations for trimming or breaking.

The centering tabs can have a round, rectangular, oval, or any other conveniently shaped cross section. They can also have a tapered profile, such that different centering tabs have different “heights” along the length of the mold. The result can be radial or axial variation of the dimension of the centering tabs, such that their geometries differ from a proximal end to a distal end. This variation can ease removal of the implant after the coating has cured. This variation in centering tab geometry can also be used to control dosage or concentration of an agent—to result in an elution profile. For example, a slightly conical tab with a flat end can include a stabilizing base within the mold material. This can provide mechanical rigidity and help prevent deflection of the mold at the tab position when the weight of the nail or other implant is placed on it, and when a mechanical force is applied as the coating is injected into the mold.

The mold can also consist of a flexible support portion provided adjacent to, and outside of, an inner portion having a different flexibility or density. In one, non-limiting example, this allows for adjustment of the resulting mold shape based upon mechanical “push back” from the variation in the nail geometry, such as in a Herzog curve area. This same result can also be achieved with other types of molds such as with the ring spacers already described above. In other instances, it may be desirable for the mold to have varying densities throughout, and have a profile such as a corrugated “bendy straw”. This allows a further ability to adapt the mold to conform to curved portions of the implant in areas that are less flexible such as adjacent the centering tabs.

Other techniques for registering the nail or other implant within the mold can include mechanical attachments positioned along the inside of the mold from the proximal and/or distal end. This can include using existing feature(s) of the nail or other implant to accept mechanical connections that suspend the nail/implant within the mold. In one example, the plugs 170, 180 may also serve to hold the nail/implant in position.

As mentioned and shown above, the plugs (170, 180, 210, 290, etc.) can also act as restrictors to prevent incursion of the coating material into the screw clearance holes and the cannula within a nail or other implant 201. These plugs can be formed of soft neoprene, or could be a hard plastic material. Their design can be limited to serving to plug the holes to the extent of the radius of the nail, or they can be longer to act as standoffs as discussed above. In some embodiments the standoffs, centering tabs, or plugs may be constructed of a bio-absorbable or dissolvable material such as PLA or the like.

In some embodiments, these plugs can have a distal feature to set the shape of an insertion edge. In particular, the plugs can be tapered to match a taper provided on the inner diameter of the ends of the mold. Provided a taper in this manner can help prevent damaging the insert when it is placed within the mold.

The tabs/plugs and associated holes can have additional attributes.
a. A mechanical feature can be provided to set a specified distance, such as from a point of insertion of the plug into the nail, to the edge of the mold.
b. Centering tabs can be provided with various diameters (for example 4 mm and 5 mm), to restively mate to a 4.5 mm lip or 5.5 mm lip on the outside of the mold clearance, each also tapering out to a 5 mm, or 6 mm diameter at the interface to the mold.
c. The interface between the plugs and the holes in the mold may include additional mold deflection features such as a surge shape to deflect the mold when it is bent.
d. Stand offs can be shaped to accommodate vendor variations (such as the precise location of the Herzog bend which may vary between manufacturers.
e. The pair of plugs 170, 180 can be independently inserted, and not connect to one another, or they can mechanically connect to each other within the hole 140 so as to provide addition mechanical support for the mold.
f. Further tabs can include more than one clearance hole of varying geometry.
g. The tapered plugs 170, 180 can be supplied as a pack with varying lengths and geometries.
h. The plugs can have an inner core of another material to prevent radial compressibility to set the mold width, and an outer softer sealing material such as silicon top, to prevent leakage of the implant cement or other material into the clearance holes.

A benefit over prior art techniques is that post-coating processes are no longer needed to drill or otherwise clean out the clearance holes, or otherwise clear debris from the cannulated areas. Such drilling generally requires using an aligning/aiming arm provided by the nail manufacturer for application of locking screws, but repurposed for “finding” the location of the clearance holes under the coating martial, and then individually drilling them out. With the designs and methods described herein, those difficulties are avoided, saving time and improving efficiency, while reducing possible debris within a nail or other device.

Additional plugs or inserts may be provided to accommodate drug delivery or other material delivery as discussed above. These may be inserted into mold and attached to either the nail or prothesis itself, or to the mold. An adhesive can be used to secure these in place, and they can implemented from PLA or other bio-absorbable materials appropriate for drug delivery.

The plugs and mold can be packaged as a kit. For example, a kit may include a mold with a 11 mm outer diameter, for use with a smaller nail like a 8 mm nail, and to provide 1.5 mm thickness radial coating. The same kit might also accommodate different nail diameters and include plus of different shapes and sizes.

Variations in the coating of a plate type insert are also possible. A bed of nails can be provided on an inner surface of a mold formed of flexible material. The coating process may then use pressure (pneumatic, hydronic, etc.), or the mold can be formed of compressible foam, to enable the mold to confirm to a uniform thickness around the plate.

The mold may be fabricated as a two part silicon mold. Such a mold can include an external outer layer, made of either a denier foam or similar material, or plastic, either flexible or rigid.

Alternatively, the mold is fabricated as a tube, either from clear or opaque material. The tube could be made from the same materials used to fabricate flexible tubes suitable for other surgical uses. In some configurations, spacers placed within such a tube could include one or more o-rings placed on the nail to provide consistency in coating thickness around any bends. These or other spacers may include dissoluble materials such as a PLA, or non-resolvable features.

A suction port can be added to a distal end of the tube to allow for drawing in of the coating material prior to curing. This can also promote speed and consistency in curing the coating.

The tube may include a mechanically pre-designed “thin wall stress riser” for easy splitting and removal of the implant after the coating(s) cure or set up.

In one embodiment, the tube may also include one or more longitudinal “fins” to aid in separating the tube using the stress risers. For example, one fin may be fabricated on each half of the tube to assist with breaking the risers. Breaking open the tube can be accomplished using the fins and a mechanical attachment point for the tube injection and/or evaluation ports, including on the ends of the tube.

The tube can be trimmable to a desired length, in some embodiments, and including variation(s) with multiple injection ports and or evacuation ports along the length of the tube. The different injection ports can provide access for bone cement/antibiotic/or other mixture of medicines or other agents.

Also, when the spacers/plugs are formed of PLA or other bio absorbable material, they can further have antibiotic or other medicine fabricated within the PLA structure (or other absorbable material).

A “pack” can be assembled to accommodate any number of different implant devices from different manufacturers. Such a pack could, for example, include two or more tubes of different diameters and/or lengths, a number of different stand off plugs of several heights or diameters, and several ejection and injection caps, including a ejection cap for different suction port.

The mold can be fabricated from a material that is rollable to provide a “condom like” mold that is rolled onto a fixation nail. The “standoffs” used in this arrangement can be inserted into existing clearance holes in the nail as described above. An adhesive can be applied to such standoffs or to the tubing or to the nail. For example, the centering tabs or rings can be stuck or adhered onto the nail before the “condom” is rolled on. Alternatively, the adhesive applied to the standoffs may be applied to mold as the mold is rolled onto the tube. Such standoffs may be applied either to the clearance hole plugs, and/or to other locations along the nail, now using adhesive applied to the nail or to the exterior of the mold. The adhesive can be swirled on or applied strategically as needed.

The rollable mold could also be used as an inner lining for an outer, more rigid, mechanically supporting mold. In this instance, the use of the inner rollable mold makes removal of the nail from the outer mold easier. It can also support better water-tight sealing of the mold, for example, to prevent bone cement or coating materials from leaking out of the larger mold.

The hole plugs/standoffs could be mechanically connected via screwing or snapping together as described above. If they are assembled by screwing them together it may be easier to remove them after the coating is cured by simply unscrewing them. A releasing agent, such as mineral spirits, or some other lubricant, may be applied to the inner area of any of the plugs or molds, described herein, so as to more easily release cured cement, or other material from the surface of the mold, after curing. The tube can also be pre-coated with a lubricant to allow any standoffs to slide down the tube when the coating process is complete.

Cement or other agents may have a stimulus-curing attribute, such that exposure to ultraviolet (UV) light or some other external stimulus cause it to cure. The stimulus can be heat, radio frequency or other electromagnetic (EM) energy, infrared energy, ultrasound, etc.

When a perforated (scored) tube as described above is utilized, a surgical method may proceed as follows. A tube suitable for matching the dimensions of the surgical implant, such as a femoral nail, is chosen. One or more plugs are inserted into openings in the nail The nail is then placed within the tube and end caps are placed on the proximate and distal ends of the tube. Coating agents are then pumped into the tube via the ports and allowed to cure. Once cured, the tube is broken open along the score line. The implant is removed from the opened tube and the plugs are removed from the implant to expose the holes. The implant is then ready for insertion into the patient.

The devices, products, methods, and processes discussed above should be considered to be examples. Various configurations may omit, substitute, or add various procedures or components as appropriate. For instance, in alternative configurations, the methods may be performed in an order different from that described, and/or various components may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Also, configurations may be described as one or more processes. Although each may describe operations as a sequential process, many of the operations can be performed in parallel or concurrently. In addition, the order of the operations may be rearranged. A process may have additional states or steps not explicitly described. It also should be understood that the components and assemblies may include more or fewer elements, be arranged differently, or be represented differently.

The above description has therefore particularly shown and described example embodiments. However, it will be understood by those skilled in the art that various changes in form and details may be made therein without departing from the legal scope of this patent as encompassed by the appended claims.

1. An intraoperative apparatus for fabrication of an implant device having a controlled configuration of a coating material, the apparatus comprising:
a mold providing a cavity for accommodating insertion of the implant device into the mold; and
one or more removable plugs, adapted to be placed into corresponding openings in the implant device, the one or more removable plugs having at least one dimension that depends on the controlled configuration of the coating material.

2. The apparatus of claim 1 wherein the one or more removable plugs further provide structural support for at least one portion of the mold.

3. The apparatus of claim 1 wherein the one or more removable plugs act as a standoff for the implant device when the implant device is inserted into the cavity, to thereby control a thickness of the coating material.

4. The apparatus of claim 1 wherein the implant device has a through hole formed therein, and the one or more removable plugs comprise a first plug portion and a second plug portion configured to mate with the first plug portion via the through hole.

5. The apparatus of claim 4 wherein the through hole is located adjacent a bend in the implant device.

6. The apparatus of claim 1 wherein the mold further includes one or more ports for injection and/or extraction of the coating material.

7. The apparatus of claim 1 wherein the coating material comprises one or more of an antibiotic, aminoglycoside, vancomycin, pain medication, chemotherapy agent, biological agent, growth factor agent, stem cell agent, ferrous material, copper, silver, thermally or non-thermally stable agent relative to polymerization.

8. The apparatus of claim 1 wherein the mold is configured to target the coating material to selected parts of the implant device.

9. The apparatus of claim 1 wherein the mold is configured to apply different densities of the coating material according to a spatial distribution across an axial or radial profile of the implant.

10. The apparatus of claim 1 wherein the implant device comprises an intramedullary nail, rod, plate, wire, arthroplasty component, screw, spacer, cage, pin, artificial joint or or other component to repair, augment, or replace a body part.

11. A method for controlled fabrication of an implant device, the method comprising:
installing one or more removable plugs into corresponding openings of the implant device, the one or more removable plugs thereby covering the openings, and the one or more removable plugs having at least one dimension that depends on a desired property of a coating material;
subsequently inserting the implant device having the one or more plugs into a mold having a cavity; and
applying the coating material to the mold, thereby coating the implant device, and with the one or more removable plugs restricting access of the coating material to the openings.

12. The method of claim 11 wherein the one or more removable plugs further provide structural support for at least one portion of the mold.

13. The method of claim 11 wherein the one or more removable plugs act as a standoff for the implant device when the implant device is inserted into the cavity, to thereby control a thickness of the coating material.

14. The method of claim 11 wherein the implant device has a through hole formed therein, and the one or more removable plugs comprise a first plug portion and a second plug portion configured to mate with the first plug portion via the through hole.

15. The method of claim 14 wherein the through hole is located adjacent a bend in the implant device.

16. The method of claim 11 wherein the mold further includes one or more ports for injection and/or extraction of the coating material.

17. The method of claim 11 wherein the coating comprises one or more of an antibiotic, aminoglycoside, vancomycin, pain medication, chemotherapy agent, biological agent, growth factor agent, stem cell agent, ferrous material, copper, silver, thermally or non-thermally stable agent relative to polymerization.

18. The method of claim 11 wherein the mold is configured to target the coating material to selected parts of the implant device.

19. The method of claim 11 wherein the mold is configured to apply different densities of the coating material according to a spatial distribution across an axial or radial profile of the implant.

20. The method of claim 11 wherein the implant device comprises an intramedullary nail, rod, plate, wire, arthroplasty component, screw, spacer, cage, pin, artificial joint or or other component to repair, augment, or replace a body part.
